(19) United States
(12) Patent Application Publication
(10) Pub. No.: US 2024/0320962 A1
(43) Pub. Date: Sep. 26, 2024

(54) SITE-SPECIFIC ADAPTATION OF AUTOMATED DIAGNOSTIC ANALYSIS SYSTEMS

(71) Applicant: Siemens Healthcare Diagnostics Inc., Tarrytown, NY (US)

(72) Inventors: Venkatesh NarasimhaMurthy, Hillsborough, NJ (US); Vivek Singh, Princeton, NJ (US); Yao-Jen Chang, Princeton, NJ (US); Benjamin S. Pollack, Jersey City, NJ (US); Ankur Kapoor, Plainsboro, NJ (US); Rayal Raj Prasad Nalam Venkat, Princeton, NJ (US)

(73) Assignee: Siemens Healthcare Diagnostics Inc., Tarrytown, NY (US)

(21) Appl. No.: 18/576,256

(22) PCT Filed: Jul. 6, 2022
(86) PCT No.: PCT/US2022/073473

§ 371 Date: Jan. 3, 2024

Related U.S. Application Data
(60) Provisional application No. 63/219,342, filed on Jul. 7, 2021.

(51) Int. Cl. G06V 10/776; G06V 10/778; G06V 10/94; G06V 20/69; G16H 50/20

(52) U.S. Cl. CPC G06V 10/776; G06V 10/778; G06V 10/945; G06V 20/698; G16H 50/20

(57) ABSTRACT

Methods of characterizing a sample container or a biological sample in an automated diagnostic analysis system using an artificial intelligence (AI) algorithm include retraining of the AI algorithm in response to characterization confidence levels determined to be unsatisfactory. The AI algorithm is retrained with data (including image data and/or non-image data) having features prevalent at the site where the automated diagnostic analysis system is operated, which were not sufficiently or at all included in training data used to initially train the AI algorithm. Systems for characterizing a sample container or a biological sample using an AI algorithm are also provided, as are other aspects.

### CROSS REFERENCE TO RELATED APPLICATION

This application claims the benefit of U.S. Provisional patent Application No. 63/219,342, entitled “SITE-SPECIFIC ADAPTATION OF AUTOMATED DIAGNOSTIC ANALYSIS SYSTEMS” filed Jul. 7, 2021, the disclosure of which is hereby incorporated by reference in its entirety for all purposes.

### FIELD

This disclosure relates to automated diagnostic analysis systems.

### BACKGROUND

In medical testing, automated diagnostic analysis systems may be used to analyze a biological sample to identify an analyte or other constituent contained in the sample. The biological sample may be, e.g., urine, whole blood, blood serum, blood plasma, interstitial liquid, cerebrospinal liquid, and the like. Such samples are usually contained in sample containers (which may also be referred to as collection tubes, test tubes, vials, etc.). Sample containers may be transported via container carriers on automated tracks to and from various imaging, processing, and analyzer stations within an automated diagnostic analysis system.

Automated diagnostic analysis systems typically include a sample pre-processing or pre-screening procedure to “characterize” various features of sample containers and/or the samples therein. Characterization (e.g., identification or classification of features) may be performed by an artificial intelligence (AI) algorithm executing on a system controller, processor, or like device of the automated diagnostic analysis system. The AI algorithm may perform “segmentation,” wherein various regions of a sample container and/or sample therein may be identified and/or classified. Characterization of a sample using an AI algorithm may also perform an HILN determination. An HILN determination identifies whether an interferent, such as hemolysis (H), icterus (I), and/or lipemia (L), which may adversely affect test results, is present in the sample to be analyzed, or whether the sample is normal (N) and can be further processed. If an interferent is present, the degree of the interferent may also be classified by the AI algorithm.

Characterization is typically performed using imaged data of the sample container and sample therein. That is, images of the sample container and sample therein may first be captured at an imaging station of the automated diagnostic analysis system, and are then analyzed using the AI algorithm.

Before the AI algorithm is used for characterization, the AI algorithm is “trained” to characterize features likely to be encountered in the imaged sample data. Training is performed by providing the AI algorithm with training data (e.g., imaged sample data) having annotated (identified) features therein. This training data may be referred to as a “ground truth.”

To ensure that automated diagnostic analysis systems perform consistently wherever deployed, the AI algorithm may be trained with a standard set of training data that includes a sampling of common features to be characterized by the AI algorithm.

The AI algorithm may, however, be unable or less likely to accurately characterize certain features or certain variations of features that may not have been included in the training data used to train the AI algorithm.

Accordingly, improved training of AI algorithms for use in automated diagnostic analysis systems is desired.

### SUMMARY

In some embodiments, a method of characterizing a sample container or a sample in an automated diagnostic analysis system is provided. The method includes capturing an image of a sample container containing a sample by using an imaging device, characterizing the image using a first artificial intelligence (AI) algorithm executing on a system controller of the automated diagnostic analysis system, determining a characterization confidence level of the image using the system controller, and triggering a retraining of the first AI algorithm with retraining data in response to a characterization confidence level determined to be below a pre-selected threshold. The triggering is initiated by the system controller, and the retraining data includes image data captured by the imaging device or non-image data that includes features prevalent at a current location of the automated diagnostic analysis system that were not sufficiently or at all included in training data used to initially train the first AI algorithm.

In some embodiments, an automated diagnostic analysis system is provided that includes an imaging device configured to capture an image of a sample container containing a sample, and a system controller coupled to the imaging device. The system controller is configured to: characterize an image captured by the imaging device using a first artificial intelligence (AI) algorithm executing on the system controller, determine a characterization confidence level of the image using the system controller, and in response to a characterization confidence level determined to be below a pre-selected threshold, trigger a retraining of the first AI algorithm. The retraining is performed by the system controller with retraining data that includes image data captured by the imaging device or non-image data that includes features prevalent at a current location of the automated diagnostic analysis system that were not sufficiently or at all included in training data used to initially train the first AI algorithm.

In some embodiments, a method of characterizing a sample container or a sample in an automated diagnostic analysis system is provided. The method includes capturing data representing a sample container containing a sample by using one or more of an optical, acoustic, humidity, liquid volume, vibration, weight, photometric, thermal, temperature, current, or voltage sensing device, characterizing the data using a first artificial intelligence (AI) algorithm executing on a system controller of the automated diagnostic analysis system, determining a characterization confidence level of the data using the system controller, and triggering a retraining of the first AI algorithm with retraining data in response to a characterization confidence level determined to be below a pre-selected threshold. The triggering is initiated by the system controller, and the retraining data includes features prevalent at a current location of the automated diagnostic analysis system that were not sufficiently or at all included in training data used to initially train the first AI algorithm.

Still other aspects, features, and advantages of this disclosure may be readily apparent from the following detailed description and illustration of a number of example embodiments and implementations, including the best mode contemplated for carrying out the invention. This disclosure may also be capable of other and different embodiments, and its several details may be modified in various respects, all without departing from the scope of the invention. For example, although the description below relates to AI algorithms used for pre-processing/pre-screening sample containers and samples therein based on imaged data, the methods and systems described herein may be readily adapted to AI algorithms for analyzing measurement results and/or other applications based on sensor, text, and/or other non-image data where features, conditions, and constraints prevalent at the site where the AI algorithm is executed were not adequately included in the original training data.

This disclosure is intended to cover all modifications, equivalents, and alternatives falling within the scope of the appended claims (see further below).

### BRIEF DESCRIPTION OF DRAWINGS

The drawings, described below, are provided for illustrative purposes, and are not necessarily drawn to scale. Accordingly, the drawings and descriptions are to be regarded as illustrative in nature, and not as restrictive. The drawings are not intended to limit the scope of the invention in any way.

FIG. 1 illustrates a top schematic view of an automated diagnostic analysis system configured to perform pre-processing/pre-screening characterization and one or more biological sample analyses according to embodiments provided herein.

FIG. 2A illustrates a side elevation view of a sample container including a separated sample containing a serum or plasma portion that may contain an interferent according to embodiments provided herein.

FIG. 2B illustrates a side view of the sample container of FIG. 2A held in an upright orientation in a holder that can be transported within the automated diagnostic analysis system of FIG. 1 according to embodiments provided herein.

FIG. 3 illustrates a block diagram of a computer for use with the automated diagnostic analysis system of FIG. 1 according to embodiments provided herein.

FIG. 4 illustrates a flowchart of a method of characterizing a sample container and/or a sample in the automated diagnostic analysis system of FIG. 1 according to embodiments provided herein.

FIG. 5 illustrates a schematic top view of a quality check station of the automated diagnostic analysis system of FIG. 1 (with chamber top removed for clarity) configured to capture images according to embodiments provided herein.

FIG. 6 illustrates block diagram of a pre-screening characterization architecture including an AI algorithm configured to perform segmentation and interferent determinations of a sample container and/or sample contained therein in the automated diagnostic analysis system of FIG. 1 according to embodiments provided herein.

### DETAILED DESCRIPTION

Automated diagnostic analysis systems described herein perform pre-processing/pre-screening characterization of sample containers and biological samples contained therein to facilitate automated container handling, to prepare samples for analysis, and to determine suitability of the samples for one or more biological analyses performed by the automated diagnostic analysis system. Characterization may include identifying and/or classifying features recognizable in captured images of sample containers and biological samples contained therein. Note that in alternative embodiments, non-image data (from, e.g., one or more sensors such as one or more, e.g., temperature sensors, acoustic sensors, humidity sensors, liquid volumes sensors, weight sensors, vibration sensors, current sensors and/or voltage sensors) and/or text data may be used as input instead of, or in addition to, captured images. Characterization of a sample container may indicate, e.g., a size and type of the container, fluid levels or volumes therein, and whether the container has a cap thereon and, if so, what type of cap. This information may be used to program robotic container handlers of the automated diagnostic analysis system to facilitate transport and positioning of the sample container and aspiration of the sample from the sample container. Characterization of a biological sample may determine, e.g., a presence and/or a degree of an interferent (e.g., hemolysis, icterus, and/or lipemia) and thus whether the biological sample is sufficient/acceptable to be further processed and analyzed.

The pre-processing/pre-screening characterization may be performed using an artificial intelligence (AI) algorithm executing on a computer (e.g., a system controller, a processor, or like device) of the automated diagnostic analysis system. The AI algorithm may be any suitable machine-learning software application capable of “learning” (i.e., reprogramming itself) as it processes more data. The AI algorithm may be trained with training data to characterize expected or common features. The training data may include images of the features to be characterized. In some embodiments, a large training dataset of images of features to be characterized may be captured in different views and/or lighting conditions by one or more imaging devices (e.g., cameras or the like). In some embodiments, the training data may additionally or alternatively include non-image data.

After pre-processing/pre-screening, sample containers and the biological samples contained therein may be transported to an appropriate analyzer station of the automated diagnostic analysis system, where the sample may be combined with one or more reagents and/or other materials in a reaction vessel. Analytical measurements may then be made via photometric or other analysis techniques. In some embodiments, the analytical measurements may be analyzed using an appropriately trained AI algorithm to determine amounts of analytes or other constituents in the samples and/or to identify one or more disease states. Although the following disclosure is described primarily with respect to AI algorithms used for pre-processing/pre-screening characterization, the methods and systems of retraining AI algorithms based on site-specific (current location) features disclosed herein may also apply to AI algorithms used for other purposes, such as, e.g., analyzing sample measurement results.

To monitor the performance of an automated diagnostic analysis system and, in particular, an AI algorithm used therein, “confidence” levels may be routinely or continuously determined by (the AI algorithm itself and/or one or more other algorithms or programs of) the automated diagnostic analysis system in accordance with one or more embodiments. The determined confidence levels indicate the likelihood that the characterizations and/or analyses performed by the AI algorithm are accurate and/or correct. In some embodiments, the determined confidence levels may be in the form of a value (e.g., between 1 and 100 or between 0.0 and 1.0) or a percentage (between 0% and 100%). Other suitable confidence measures may be used. Low confidence levels, below a predetermined threshold, may be indicative of insufficient training of the AI algorithm.

For example, low characterization confidence levels may result from operating an automated diagnostic analysis system in a current location (including a particular geographical region) or in a particular manner (e.g., performing a specialized type of diagnostic analysis relevant to the particular geographical region) where certain features or variations of features are unique or more prevalent than the features included in the training data that was used to initially train an AI algorithm of the automated diagnostic analysis system. Low characterization confidence levels may also result after operating an automated diagnostic analysis system for a period of time where, e.g., new or varied types of sample containers may begin to be used and/or new or varied features of biological samples may appear because of a seasonal or regional disease outbreak.

In cases where low confidence levels are determined, it may be desirable to retrain the AI algorithm. The process of retraining AI algorithms in conventional systems may be relatively cumbersome and manually intensive. For example, in some conventional systems, deficiencies in an AI algorithm may not be identified until the system encounters a malfunction (e.g., confidence levels may not be routinely determined during operation). Troubleshooting incorrect test results can be manually time consuming and costly, particularly when a system is be taken offline due to a malfunction. Upon identification of an AI algorithm's deficiencies as the cause, retraining data can be collected (also usually a manual task) and forwarded to engineering teams of the manufacturer of the diagnostic system. The AI algorithm may then be retrained at the manufacturer and returned for reloading into the system at the user's site. Plainly, conventional retraining processes may be very expensive and time consuming.

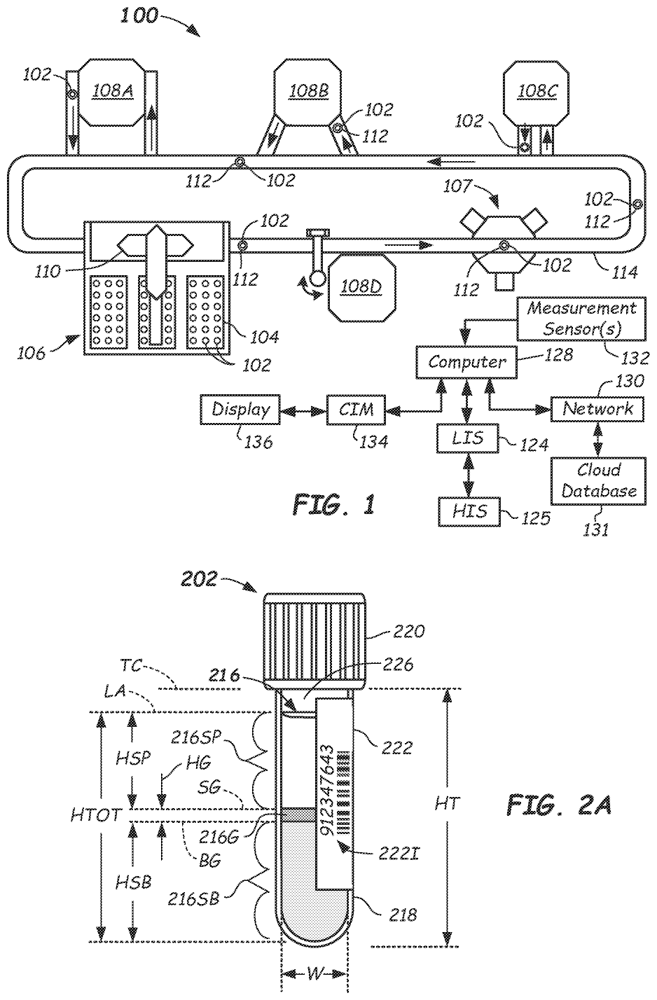

In accordance with one or more embodiments, improved automated diagnostic analysis systems and methods of characterizing a sample container or a sample in an automated diagnostic analysis system will be explained in greater detail below in connection with FIGS. 1-6. The improved systems and methods may include monitoring of AI algorithm performance, collection, and annotation of site-specific data for retraining, and/or retraining of the AI algorithm at the site (current location) where the automated diagnostic analysis system is operated.

FIG. 1 illustrates an automated diagnostic analysis system 100 according to one or more embodiments. Automated diagnostic analysis system 100 may be configured to automatically characterize, process, and/or analyze biological samples contained in sample containers 102. Sample containers 102 may be received at system 100 in one or more racks 104 provided at a loading area 106 prior to transportation to, characterization at quality check station 107, and analysis at one or more analyzer stations 108A-D of system 100.

At least one of analyzer stations 108A-D (e.g., analyzer station 108D) may perform pre-processing and may include, e.g., a centrifuge to separate various components of a biological sample and/or a decapper for removing a cap from a sample container 102. One or more analyzer stations 108A-D may include one or more clinical chemistry analyzers, assaying instruments, and/or the like, and may be used to analyze for chemistry or assay for the presence, amount, or functional activity of a target entity (an analyte), such as, e.g., DNA or RNA. Analytes commonly tested for in clinical chemistry analyzers include chemical components such as metabolites, antibodies, enzymes, hormones, lipids, substrates, electrolytes, specific proteins, abused drugs, and therapeutic drugs. More or less numbers of analyzer stations 108A-D may be used in system 100.

A robotic container handler 110 may be provided at loading area 106 to grasp a sample container 102 from the one or more racks 104 and load the sample container 102 into a container carrier 112 positioned on a track 114, via which sample containers 102 may be transported throughout system 100.

Sample containers 102 may be any suitable containers, including transparent or translucent containers, such as a blood collection tubes, test tubes, sample cups, cuvettes, or other containers capable of containing and allowing the biological samples contained therein to be imaged. Sample containers 102 may be varied in size and may have different types of caps and/or cap (indicator) colors.

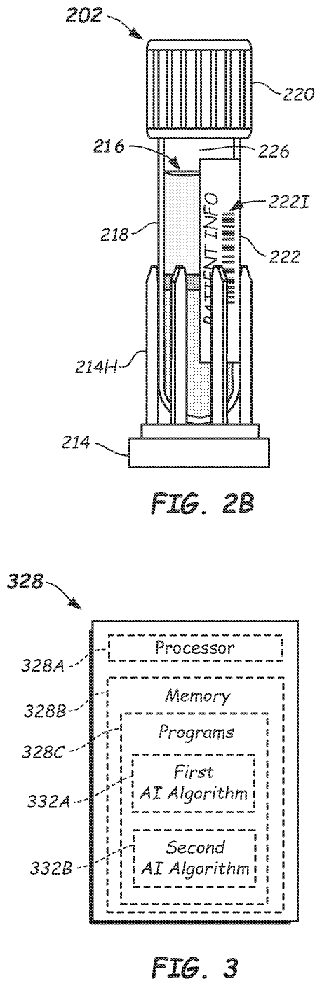

FIGS. 2A and 2B illustrate an embodiment of a sample container and a biological sample located therein. Sample container 202 may be representative of sample containers 102 (FIG. 1) and biological sample 216 may be representative of samples located in sample containers 102. Sample container 202 may include a tube 218 and may be capped with a cap 220. Caps on different sample containers may be of different types and/or colors (e.g., red, royal blue, light blue, green, grey, tan, yellow, or color combinations), which may indicate, e.g., specific tests sample container 202 is used for, a type of additive included therein, whether the sample container includes a gel separator, etc. In some embodiments, the cap type may be identified by a characterization of sample container 202, as described further below.

Sample container 202 may be provided with at least one label 222 that may include identification information 222I (i.e., indicia) thereon, such as a barcode, alphabetic characters, numeric characters, or combinations thereof. Identification information 222I may include or be associated with patient information via a laboratory information system database (e.g., LIS 124 of FIG. 1). The database may include patient information (referred to as text data) such as patient name, date of birth, address, heath conditions, or diseases, and/or other personal information as described herein. The database may also include other text data, such as tests to be performed on sample 216, the time and date sample 216 was obtained, medical facility information, and/or tracking and routing information. Other text data may also be included.

The identification information 222I may be machine readable and darker (e.g., black) than the label material (e.g., white paper) so that the identification information 222I can be readily imaged or scanned. The identification information 222I may indicate or may otherwise be correlated via the LIS or other test ordering system to a patient's identification as well as tests to be performed on sample 216. The identification information 222I may be provided on label 222, which may be adhered to or otherwise provided on an outside surface of tube 218. In some embodiments, label 222 may not extend all the way around the sample container 202 or along a full length/height of the sample container 202.

Sample 216 may include a serum or plasma portion 216SP and a settled blood portion 216SB contained within tube 218. A gel separator 216G may be located between the serum or plasma portion 216SP and the settled blood portion 216SB. Air 226 may be above the serum and plasma portion 216SP. A line of demarcation between the serum or plasma portion 216SP and air 226 is defined as the liquid-air interface LA. A line of demarcation between the serum or plasma portion 216SP and the gel separator is defined as a serum-gel interface SG. A line of demarcation between the settled blood portion 216SB and the gel separator 216G is defined as a blood-gel interface BG. An interface between air 226 and cap 220 is defined as a tube-cap interface TC.

The height of the tube HT is defined as a height from a bottom-most part of tube 218 to a bottom of cap 220 and may be used for determining tube size (e.g., tube height and/or tube volume). A height of the serum or plasma portion 216SP is HSP and is defined as a height from a top of the serum or plasma portion 216SP at LA to a top of the gel separator 216G at SG. A height of the gel separator 216G is HG and is defined as a height between SG and BG. A height of the settled blood portion 216SB is HSB and is defined as a height from the bottom of the gel separator 216G at BG to a bottom of the settled blood portion 216SB. HTOT is a total height of the sample 216 and equals the sum of HSP, HG, and HSB. The width of the cylindrical portion of the inside of the tube 218 is W. An AI algorithm (as described below) may determine one or more of the above-described dimensions as part of a segmentation characterization performed at quality check station 107 in automated diagnostic analysis system 100.

FIG. 2B illustrates sample container 202 located in a carrier 214. Carrier 214 may be representative of carriers 114 of FIG. 1. Carrier 214 may include a holder 214H configured to hold sample container 202 in a defined upright position and orientation. Holder 214H may include a plurality of fingers or leaf springs that secure sample container 202 in carrier 214, some of which may be moveable or flexible to accommodate different sizes (widths) of sample container 202. In some embodiments, carrier 214 may be transported from loading area 106 of FIG. 1 after being offloaded from one of racks 104 by robotic container handler 110.

Returning to FIG. 1, automated diagnostic analysis system 100 may include a computer 128 or, alternatively, may be configured to communicate remotely with an external computer 128. Computer 128 may be, e.g., a system controller or the like, and may have a microprocessor-based central processing unit (CPU). Computer 128 may include suitable memory, software, electronics, and/or device drivers for operating and/or controlling the various components (including quality check station 107 and analyzer stations 108A-D) of system 100. For example, computer 128 may control movement of carriers 112 to and from loading area 106, about track 114, to and from quality check station 107 and analyzer stations 108A-D, and to and from other stations and/or components of system 100. One or more of quality check station 107 and analyzer stations 108A-D may be directly coupled to computer 128 or in communication with computer 128 through a network 130, such as a local area network (LAN), wide area network (WAN), or other suitable communication network, including wired and wireless networks. Computer 128 may be housed as part of system 100 or may be remote therefrom.

In some embodiments, computer 128 may be coupled to a computer interface module (CIM) 134. CIM 134 and/or computer 128 may be coupled to a display 136, which may include a graphical user interface. CIM 134, in conjunction with display 136, enables a user to access a variety of control and status display screens and to input data into computer 128. These control and status display screens may display and enable control of some or all aspects of quality check station 107 and analyzer stations 108A-D for preparing, pre-screening (characterizing), and analyzing sample containers 102 and/or the samples located therein. CIM 134 may be used to facilitate interactions between a user and system 100. Display 136 may be used to display a menu including icons, scroll bars, boxes, and buttons through which a user (e.g., a system operator) may interface with system 100. The menu may include a number of functional elements programmed to display and/or operate functional aspects of system 100.

FIG. 3 illustrates a computer 328, which may be a system controller of automated diagnostic analysis system 100 and an embodiment of computer 128. Computer 328 may include a processor 328A and a memory 328B, wherein processor 328A is configured to execute programs 328C stored in memory 328B. Programs 328C may operate components of automated diagnostic analysis system 100 and may further perform characterizations and/or retraining of AI algorithms as described herein. One or more of programs 328C may be artificial intelligence (AI) algorithms that characterize, process, and/or analyze image data and other types of data (e.g., non-image data (e.g., sensor data) and/or text data). In some embodiments, memory 328B may store a first AI algorithm 332A and a second AI algorithm 332B.

First AI algorithm 332A and second AI algorithm 332B are each executable by processor 328A and may be implemented in any suitable form of artificial intelligence programming including, but not limited to, neural networks, including convolutional neural networks (CNNs), deep learning networks, regenerative networks, and other types of machine learning algorithms or models. Note, accordingly, that first AI algorithm 332A and second AI algorithm 332B are not, e.g., simple lookup tables. Rather, first AI algorithm 332A and second AI algorithm 332B may each be trained to recognize a variety of different imaged features and each are capable of improving (making more accurate determinations or predictions) without being explicitly programmed. In some embodiments, first AI algorithm 332A and second AI algorithm 332B may each perform different tasks. For example, first AI algorithm 332A may be configured to perform characterizations of a sample container and/or a sample in automated diagnostic analysis system 100 as described herein, and second AI algorithm 332B may be configured to analyze sample measurement results. In other embodiments, first AI algorithm 332A may be an AI algorithm initially provided with system 100, and second AI algorithm 332B may be a retrained version of first AI algorithm 332A.

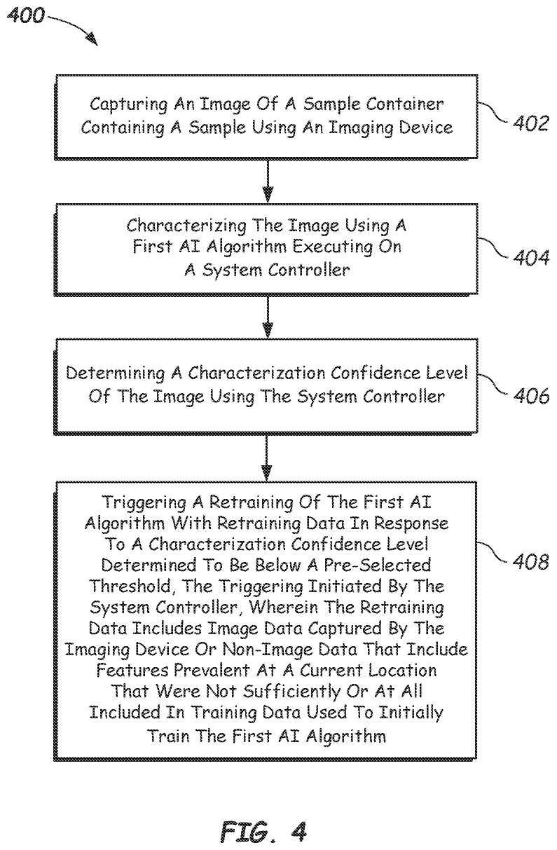

FIG. 4 illustrates a method 400 of characterizing a sample container and/or a sample in an automated diagnostic analysis system according to one or more embodiments. For example, sample container 102 or 202 and/or sample 216 may be characterized at quality check station 107 of automated diagnostic analysis system 100.

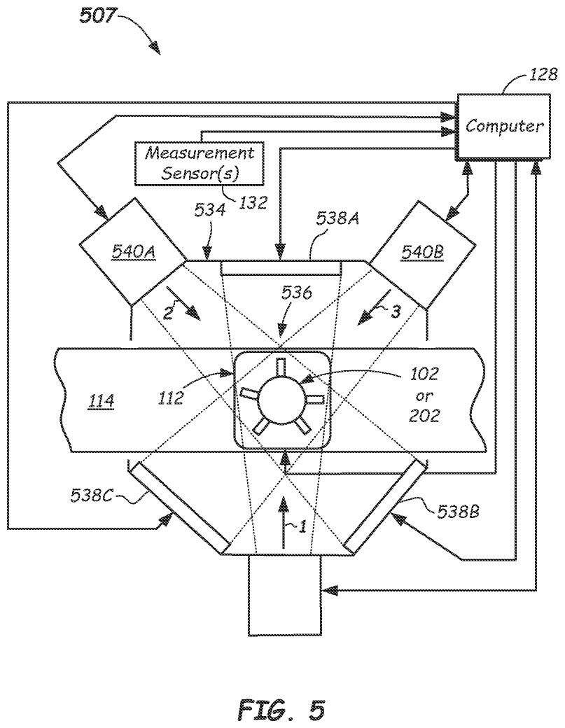

At process block 402, method 400 may begin by capturing an image of a sample container containing a sample by using an imaging device. For example, capturing an image of a sample container may be performed at quality check station 107 of automated diagnostic analysis system 100 as described in more detail in connection with FIG. 5.

FIG. 5 illustrates a quality check station 507, which may be representative of quality check station 107, according to one or more embodiments. Quality check station 507 may perform pre-screening of samples and/or sample containers based on images captured therewith. Quality check station 507 may include a housing 534 that may at least partially surround or cover track 114 to minimize outside lighting influences. Sample container 102 or 202 may be located inside housing 534 and positioned in carrier 112 at an imaging location 536 during an image-capturing sequence. Housing 534 may include one or more openings or doors (not shown) to allow carrier 112 to enter into and/or exit from quality check station 507 via track 114.

Quality check station 507 may also include one or more light sources 538A, 538B, and/or 538C that are configured to illuminate sample container 102 or 202 and/or sample 216 during the image capturing sequence. Light sources 538A, 538B, and/or 538C may be controlled (e.g., on/off and optionally brightness level) by computer 128, but may also be able to illuminate with different wavelengths of light.

Quality check station 507 may further include one or more imaging devices 540A, 540B, and/or 540C, which may be any suitable device configured to capture digital images. In some embodiments, each of imaging devices 540A, 540B, and/or 540C may be a conventional digital camera capable of capturing pixelated images, a charged coupled device (CCD), an array of photodetectors, one or more CMOS sensors, or the like. In some embodiments, the size of the captured images may be about 2560×694 pixels. In other embodiments, the size may be about 1280×387 pixels. Captured images may have other suitable pixel sizes.

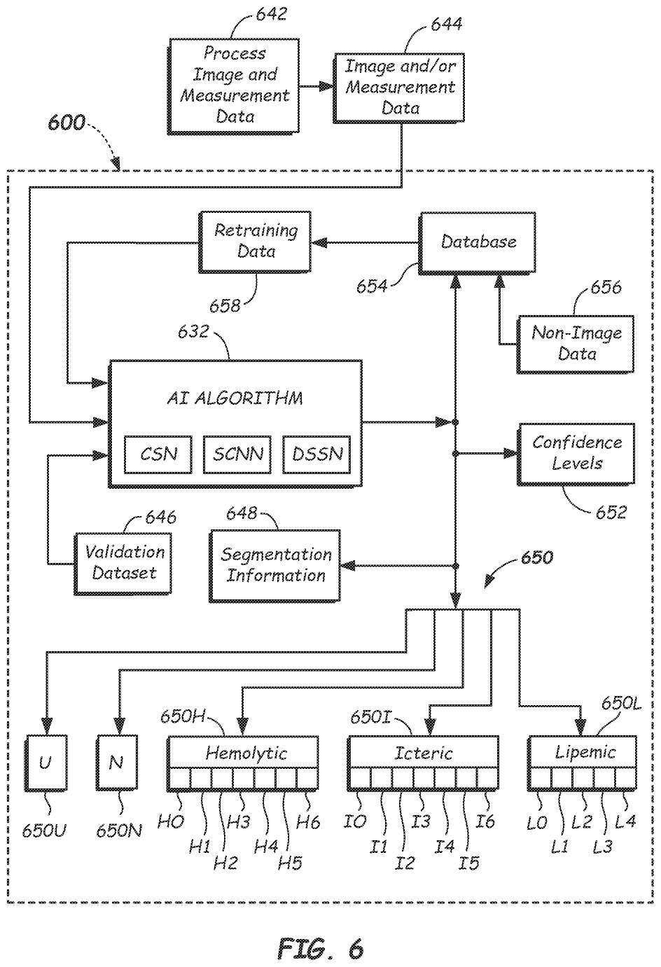

Each of imaging devices 540A, 540B, and 540C may be positioned to capture images of sample container 102 or 202 and sample 216 at imaging location 536 from a different viewpoint (e.g., viewpoints labeled 1, 2, and 3). While three imaging devices 540A, 540B, and/or 540C are shown, optionally, two, four, or more imaging devices may be used. Viewpoints 1-3 may be arranged approximately equally spaced from one another, such as about 120° apart, as shown. The images may be captured in a round robin fashion, e.g., one or more images from viewpoint 1 followed sequentially by one or more images from viewpoints 2 and 3. Other sequences of capturing images may be used, and other arrangements of imaging devices 540A, 540B, and/or 540C may be used. Each of imaging devices 540A, 540B, and/or 540C may be triggered by triggering signals generated by computer 128. Each of the captured images may be processed by computer 128 as described further below in connection with FIG. 6.

Returning to FIG. 4, method 400 may include at process block 404 characterizing the image using a first AI algorithm executing on a system controller of the automated diagnostic analysis system. For example, characterization of the image may be performed by first AI algorithm 332A executing on computer 128. Characterization of the image may facilitate handling of the sample container within automated diagnostic analysis system 100 and/or may determine whether the quality of the sample is suitable for analysis by one or more of analyzer stations 108A-D of system 100.

More particularly, characterization may provide segmentation data, which may identify various regions (areas) of a sample container and sample, such as a serum or plasma portion, a settled blood portion, a gel separator (if used), an air region, one or more label regions, a type of specimen container (indicating, e.g., height and width or diameter), and/or a type and/or color of a sample container cap. Segmentation data may include certain physical dimensional characteristics of a sample container and sample. For example, dimensions and/or locations of TC, LA, SG, BG, HSP, HSB, HT, W, and/or HTOT of sample container 202 and sample 216 (of FIGS. 2A and 2B) may be determined. Also, one or more volumes such as, e.g., serum or plasma portion 216SP and/or settled blood portion 216SB may be estimated. Other quantifiable features may also be determined.

Characterization may also provide information regarding the presence of, and optionally a degree of, an interferent (e.g., hemolysis (H), icterus (I), and/or lipemia (L)) in sample 216, or whether the sample is normal (N), prior to analysis by one or more analyzers stations 108A-D (of FIG. 1). Pre-screening in this manner may allow for additional processing where necessary and/or discarding and/or redrawing of a sample without wasting valuable analyzer resources by possibly having the presence of a sufficient amount of an interferent adversely affect the test results.

FIG. 6 illustrates a pre-screening characterization architecture 600 that includes an AI algorithm 632, which may be representative of first AI algorithm 332A, according to one or more embodiments. Pre-screening characterization architecture 600 may be implemented in quality check station 107 and/or 507 and may be controlled by computer 128 or 328 (and programs 328C). At functional block 642, raw images captured by imaging devices 540A, 540B, and/or 540C and/or measurement data from measurement sensors 132 may be processed and/or consolidated by programs 328C executed on computer 128 to produce image and/or measurement data 644. Image date may be optimally exposed and normalized image data. In some embodiments, the raw images may be processed and consolidated as described in Wissmann et al. U.S. Patent Application Publication 2019/0041318. Image data may be input to pre-screening characterization architecture 600 and more particularly to AI algorithm 632.

In other embodiments, the raw image and/or measurement data may be input directly to pre-screening characterization architecture 600 and AI algorithm 632. In still other embodiments, alternative or additional data may be processed and/or consolidated at functional block 642 by programs 328C executed on computer 128. The alternative or additional data may include measurement data generated by measurement sensors 132 of the system 100 including, but not limited to, optical, acoustic, humidity, liquid volume, vibration, weight, photometric, thermal, temperature, current, or voltage sensing device(s). In still other embodiments, alternative or additional data may be text data.

Thus, image and/or measurement data 644 may include, e.g., 1D/2D/3D sensor images and alternatively or additionally measurement data such as univariate or multivariate time series data, text labels, or system logs.

Pre-screening characterization architecture 600 may be configured to perform characterizations, such as segmentation and/or HILN determinations as described above, on image and/or measurement data 644 using AI algorithm 632. AI algorithm 632 may be factory trained with a standard set of training data that includes a sampling of common features to be characterized. AI algorithm 632 may then be validated with a validation dataset 646 before automated diagnostic analysis system 100 is put into service. The validation dataset 646 ensures that AI algorithm 632 performs as expected for input like the validation dataset and that automated diagnostic analysis system 100 meets regulatory criteria where required.

In some embodiments, the validation dataset 646 may be included with automated diagnostic analysis system 100 (e.g., stored in memory 328B of computer 328). In other embodiments, validation dataset 646 may be stored and/or executed remotely, such as in a cloud server accessible by automated diagnostic analysis system 100 via, e.g., network 130 (of FIG. 1). Validation dataset 646 may also be used to validate a retrained AI algorithm 632, as described further below.

In some embodiments, AI algorithm 632 may perform pixel-level classification and may provide a detailed characterization of one or more of the captured images. AI algorithm 632 may include, e.g., one or more of a front-end container segmentation network (CSN), a segmentation convolutional neural network (SCNN), and/or a deep semantic segmentation network (DSSN). Algorithm 632 may additionally or alternatively include other types of networks to provide segmentation and/or HILN determinations.

The CSN may be configured to output segmentation information 648 based on images of a sample container and/or a sample contained therein. Segmentation information 648 may include identification of various regions of the sample container and sample, a type of sample container (indicating, e.g., height and width or diameter), a type and/or color of a sample container cap, and/or various physical dimensional characteristics of the sample container and sample contained therein, as described above.

The SCNN and/or DSSN may output interferent classifications 650. In some embodiments, the SCNN and/or DSSN may be operative to assign a classification index to each pixel of an image based on the appearance of each pixel. Pixel index information may be further processed by the SCNN and/or DSSN to determine a final classification index for a group of pixels representing a sample. In some embodiments, only a classification index may be output, which indicates either a presence of a particular interferent, a normal (N) sample (e.g., no detectable interferent), or an un-centrifuged (U) sample (which may require centrifuging before any further processing). For example, interferent classifications 650 may include an un-centrifuged class 650U, a normal class 650N, a hemolytic class 650H, an icteric class 650I, and a lipemic class 650L. In some embodiments, the SCNN and/or DSSN may provide an estimate of the degree of an identified interferent. For example, in some embodiments, the hemolytic class 650H may include sub-classes H0, H1, H2, H3, H4, H5, and H6. The icteric class 650I may include sub-classes 10, 11, 12, 13, 14, 15, and 16. And the lipemic class 650L may include sub-classes L0, L1, L2, L3, and L4. Each of hemolytic class 650H, icteric class 650I, and/or lipemic class 650L may have other numbers of fine-grained sub-classes.

The SCNN and/or the DSSN may each include, in some embodiments, greater than 100 operational layers including, e.g., BatchNorm, ReLU activation, convolution (e.g., 2D), dropout, and deconvolution (e.g., 2D) layers to extract features, such as simple edges, texture, and parts of the serum or plasma portion and label-containing regions of images. Top layers, such as fully convolutional layers, may be used to provide correlation between the features. The output of the layer may be fed to a SoftMax layer, which produces an output on a per pixel (or per superpixel (patch)—including n×n pixels) basis concerning whether each pixel or patch includes HIL, is normal, or is un-centrifuged. In some embodiments, the CSN may have a similar network structure as the SCNN and/or DSSN, but with fewer layers.

Returning to FIG. 4 at process block 406, method 400 may include determining a characterization confidence level of the image using the system controller. A characterization confidence level indicates a probability or likelihood that the first AI algorithm has correctly identified a feature in the captured image. In other words, the characterization confidence level indicates how closely a feature in a captured image matches in appearance a feature in the training data that the first AI algorithm has determined is most likely to be the same feature. For example, a characterization confidence level of 50 (on a scale of 0-100) or 0.5 (on a scale of 0.0-1.0) indicates that the first AI algorithm's identification of a feature in a captured image has a 50% probability of being correct. Similarly, a characterization confidence level of 90 or 0.9 indicates that identification of a feature in a captured image has a 90% probability of being correct. And a confidence level of zero indicates that the first AI algorithm was not able to identify one or more features in a captured image.

Referring again to FIG. 6, characterization confidence levels 652 may be generated by AI algorithm 632 executing on computer 128 or 328 using various known techniques to quantify how closely the appearance of an identified feature in a captured image matches a feature in the training data. Alternatively, characterization confidence levels may be determined by other AI algorithms or programs that may be stored, e.g., in memory 328B and executed by computer 128 or 328 as a subroutine of AI algorithm 632.

At process block 408 of FIG. 4, method 400 may include triggering a retraining of the first AI algorithm with retraining data in response to a characterization confidence level determined to be below a pre-selected threshold, wherein the triggering can be initiated by the system controller. The retraining data includes image data captured by the imaging device that includes features prevalent at a current location of the automated diagnostic analysis system that were not sufficiently or at all included in training data used to initially train the first AI algorithm.

In some embodiments, the pre-selected threshold may be, e.g., 0.7 or greater (on a scale of 0.0-1.0), which indicates that the characterization is likely correct. In other embodiments, the pre-selected threshold may be, e.g., 0.9 or greater to provide more confidence that the characterization is correct. The pre-selected threshold may be determined by a user or based on regulatory requirements in a geographical region where the automated diagnostic analysis system is currently located and operated.

Characterized features having a confidence level below the pre-selected threshold may be automatically flagged by the system controller. For example, referring back to FIGS. 1, 3, and 6, computer 128 or 328 may automatically flag characterized features having confidence levels below the pre-selected threshold and store their corresponding captured image in a local database 654 located at the current location, which may be a part of, e.g., memory 328B. Alternatively, characterized images having confidence levels below the pre-selected threshold may be stored in a cloud database 131 accessible via network 130.

The stored images having characterized features with confidence levels below the pre-selected threshold (referred to hereinafter as “low confidence characterized images”) are likely to include sample container features and/or sample features and/or variations thereof that are prevalent at the current geographical location (current location) where the automated diagnostic analysis system 100 is operating, but were not sufficiently or at all included in training data used to initially train the first AI algorithm. For example, sample containers used at the current geographical location where the automated diagnostic analysis system 100 is operating may include container configurations or types having sizes and/or shapes that were not sufficiently or at all included in the training data used to initially train the first AI algorithm. Similarly, biological samples collected from the geographical location where the system is operating may include HILN sub-classes that were not sufficiently or at all included in the training data used to initially train the first AI algorithm.

In addition to low confidence characterized images being stored in database 654 of FIG. 6, non-image data 656 may also be stored in database 654. Non-image data 656 may be related to the current geographical location where the automated diagnostic analysis system 100 is operating. Such non-image data 656 may include, e.g., sensor data, text data, and/or user entered data. Sensor data may include data measured by and received from, e.g., one or more measurement sensors 132, such as temperature sensors, acoustic sensors, humidity sensors, liquid volume sensors, weight sensors, vibration sensors, current sensors, voltage sensors, and other sensors related to the operation of automated diagnostic analysis system 100 at the current location. Text data may be related to the low confidence characterized images and/or may include self-evaluation and analysis reports of the characterization performed by AI algorithm 632. Text data alternatively or additionally may indicate, e.g., tests being performed (e.g., assay types), patient information (e.g., age, symptoms, etc.), date of tests, time of tests, system logs (e.g., system status), and any other data related to the tests being performed by automated diagnostic analysis system 100. Some of non-image data 656 may be automatically generated by computer 128 or 328, e.g., at the same time image data is generated and/or during or after characterization. Some of non-image data 656 may also be manually generated and entered by a user via CIM 134 (of FIG. 1) or test or patient information accessed from the LIS 124 or hospital information system (HIS) 125.

In some embodiments, method 400 may include automatically annotating the stored low confidence characterized images via the system controller. For example, referring to FIGS. 1, 3, and 6, automated diagnostic analysis system 100 may automatically annotate the stored low confidence characterized images via computer 128 or 328. Additionally or alternatively, manual annotation of the low confidence characterized images may be performed by a user via CIM 134 (of FIG. 1). The annotated low confidence characterized images and, in some embodiments, some of non-image data 654, may form or be identified by computer 128 or 328 as retraining data 658, which is to be used for retraining AI algorithm 632.

In some embodiments, method 400 may include automatically retraining the first AI algorithm with the retraining data via the system controller operating in a background mode. For example, in some embodiments, AI algorithm 632 may be retrained with training data 658 via computer 128 or 328 operating in a background mode while automated diagnostic analysis system 100 continues operating with AI algorithm 632. The resulting retrained AI algorithm 632 may be stored in memory 328B as second AI algorithm 332B. The retrained algorithm may then be validated using validation dataset 646.

In some embodiments of method 400, retraining of the first AI algorithm may be automatically triggered by the system controller upon each occurrence of a determined confidence level being below a pre-selected threshold, wherein the automated diagnostic analysis system operates in a continuous or continual retraining mode.

In other embodiments, method 400 may include first notifying a user via a user interface of the automated diagnostic analysis system that the first AI algorithm is to be retrained with the retraining data in response to a characterization confidence level determined to be below a pre-selected threshold. In response to the notification during a pre-determined time period, the user may delay the retraining by replying as such via the user interface. If the user does not reply within the pre-determined time period, the retraining commences automatically.

In still other embodiments of method 400, retraining may be automatically triggered upon a certain number of low confidence characterized images being flagged and stored (e.g., in database 654). In other embodiments, retraining may be automatically triggered after a pre-specified period of system operating time (e.g., a few days or 1-2 weeks) or upon a pre-specified number of sample containers/samples having been characterized after the determination of a first low confidence characterized image. Other criteria based on determined characterization confidence levels below a pre-selected threshold may be used to automatically trigger a retraining of the first AI algorithm.

In some embodiments, after retraining the first AI algorithm to produce a second AI algorithm, method 400 may further include process blocks (not shown) that include automatically replacing the first AI algorithm with the second AI algorithm. In other embodiments, method 400 may include reporting availability of the second AI algorithm to a user via the user interface and replacing the first AI algorithm with the second AI algorithm in response to user input received via the user interface. Should the second AI algorithm not perform as expected, or perform worse than the first AI algorithm, the user may then implement replacement of the second AI algorithm with the first AI algorithm via the user interface (e.g., using CIM 134). For example, upon retraining AI algorithm 632, then validating the retrained AI algorithm 632 with validation dataset 646, and storing the retrained AI algorithm 632 as second algorithm 332B in memory 328B, computer 128 or 328 may report to a user via CIM 134 and display 136 that second algorithm 332B is available for use in pre-screening characterization architecture 600. The user may then replace AI algorithm 632 with second algorithm 332B via CIM 134. The original AI algorithm 632 (which may be stored as first AI algorithm 332A) remains stored and available should second algorithm 332B not perform as expected and need to be replaced with first AI algorithm 332A (the original AI algorithm 632).

While this disclosure is susceptible to various modifications and alternative forms, specific method and apparatus embodiments have been shown by way of example in the drawings and are described in detail herein. It should be understood, however, that the particular methods and apparatus disclosed herein are not intended to limit the disclosure or the following claims.

What is claimed is:

1. A method of characterizing a sample container or a sample in an automated diagnostic analysis system, comprising:
capturing an image of a sample container containing a sample by using an imaging device;
characterizing the image using a first artificial intelligence (AI) algorithm executing on a system controller of the automated diagnostic analysis system;
determining a characterization confidence level of the image using the system controller; and
triggering a retraining of the first AI algorithm with retraining data in response to a characterization confidence level determined to be below a pre-selected threshold, the triggering initiated by the system controller, wherein:
the retraining data includes image data captured by the imaging device or non-image data that includes features prevalent at a current location of the automated diagnostic analysis system that were not sufficiently or at all included in training data used to initially train the first AI algorithm.

2. The method of claim 1, wherein the triggering further comprises:
notifying a user via a user interface of the automated diagnostic analysis system in response to a characterization confidence level determined to be below a pre-selected threshold, wherein the notification indicates that the first AI algorithm is to be retrained with the retraining data, the triggering initiated by the system controller; and
delaying retraining of the first AI algorithm with the retraining data in response to receiving user input to delay the retraining.

3. The method of claim 1, wherein the characterizing comprises determining a presence of hemolysis, icterus, or lipemia in the sample contained in the sample container imaged by the imaging device.

4. The method of claim 1, wherein the characterizing comprises determining whether a cap is present on a sample container imaged by the imaging device.

5. The method of claim 1, further comprising storing captured images that have a determined characterization confidence level below the pre-selected threshold.

6. The method of claim 1 wherein the features prevalent at the current location of the automated diagnostic analysis system include sample container configurations or types not sufficiently or at all included in the training data used to initially train the first AI algorithm.

7. The method of claim 1 wherein the features prevalent at the current location of the automated diagnostic analysis system include sample HILN sub-classes not sufficiently or at all included in the training data used to initially train the first AI algorithm.

8. The method of claim 1 wherein the retraining data has annotations automatically generated by the system controller or is manually annotated by a user.

9. The method of claim 1 wherein the retraining data additionally includes data provided by a user via a user interface of the automated diagnostic analysis system.

10. The method of claim 1 wherein retraining the first AI algorithm produces a second AI algorithm, the method further comprising validating the second AI algorithm with a validation dataset.

11. The method of claim 1, wherein retraining the first AI algorithm produces a second AI algorithm, the method further comprising reporting availability of the second AI algorithm to a user via a user interface of the automated diagnostic analysis system.

12. The method of claim 1, wherein retraining the first AI algorithm produces a second AI algorithm, the method further comprising replacing the first AI algorithm with the second AI algorithm in response to user input received via a user interface of the automated diagnostic analysis system.

13. The method of claim 12, further comprising replacing the second AI algorithm with the first AI algorithm in response to further user input received via the user interface.

14. An automated diagnostic analysis system, comprising:
an imaging device configured to capture an image of a sample container containing a sample; and
a system controller coupled to the imaging device, the system controller configured to:
characterize an image captured by the imaging device using a first artificial intelligence (AI) algorithm executing on the system controller;
determine a characterization confidence level of the image using the system controller; and
in response to a characterization confidence level determined to be below a pre-selected threshold, trigger a retraining of the first AI algorithm performed by the system controller with retraining data that includes image data captured by the imaging device or non-image data that includes features prevalent at a current location of the automated diagnostic analysis system that were not sufficiently or at all included in training data used to initially train the first AI algorithm.

15. The automated diagnostic analysis system of claim 14, wherein the system controller is further configured to:
notify a user via a user interface of the automated diagnostic analysis system that the first AI algorithm is to be retrained with the retraining data in response to the trigger; and
delay the retraining of the first AI algorithm in response to receiving user input within a pre-determined time period to delay the retraining.

16. The automated diagnostic analysis system of claim 14, wherein the system controller is further configured to store in a storage device of the automated diagnostic analysis system captured images that have a determined characterization confidence level below the pre-selected threshold.

17. The automated diagnostic analysis system of claim 14, wherein the features prevalent at the current location of the automated diagnostic analysis system include:
sample container configurations or types not sufficiently or at all included in the training data used to initially train the first AI algorithm; or
sample HILN sub-classes not sufficiently or at all included in the training data used to initially train the first AI algorithm.

18. The automated diagnostic analysis system of claim 14, wherein the retraining of the first AI algorithm produces a second AI algorithm, and the system controller is further configured to validate the second AI algorithm with a validation dataset.

19. The automated diagnostic analysis system of claim 14, wherein the retraining of the first AI algorithm produces a second AI algorithm, and the system controller is further configured to report availability of the second AI algorithm to a user via a user interface of the automated diagnostic analysis system.

20. The automated diagnostic analysis system of claim 14, wherein the retraining of the first AI algorithm produces a second AI algorithm, and the system controller is further configured to replace the first AI algorithm with the second AI algorithm in response to user input received via a user interface of the automated diagnostic analysis system.

21. The automated diagnostic analysis system of claim 14, wherein the non-image data is received from one or more measurement sensors at the current location.

22. The automated diagnostic analysis system of claim 21, wherein the one or more measurement sensors are one or more, temperature sensors, acoustic sensors, humidity sensors, liquid volumes sensors, weight sensors, vibration sensors, current sensors or voltage sensors.

23. The automated diagnostic analysis system of claim 14, wherein the non-image data that includes the features prevalent at the current location is text data.

24. The automated diagnostic analysis system of claim 23, wherein the text data is self-evaluation and analysis reports of the characterization performed by the first AI algorithm, data related to tests being performed, or patient information.

25. A method of characterizing a sample container or a sample in an automated diagnostic analysis system, comprising:
capturing data representing a sample container containing a sample by using one or more of an optical, acoustic, humidity, liquid volume, vibration, weight, photometric, thermal, temperature, current, or voltage sensing device;
characterizing the data using a first artificial intelligence (AI) algorithm executing on a system controller of the automated diagnostic analysis system;
determining a characterization confidence level of the data using the system controller; and
triggering a retraining of the first AI algorithm with retraining data in response to a characterization confidence level determined to be below a pre-selected threshold, the triggering initiated by the system controller, wherein:
the retraining data includes features prevalent at a current location of the automated diagnostic analysis system that were not sufficiently or at all included in training data used to initially train the first AI algorithm.
